(19) United States
(12) Patent Application Publication
(10) Pub. No.: US 2016/0158196 A1
(43) Pub. Date: Jun. 9, 2016

(54) A MODIFIED-RELEASE ORAL PHARMACEUTICAL FORMULATION CONTAINING GLICLAZIDE

(71) Applicant: VALPHARMA INTERNATIONAL S.P.A., Pennabilli (IT)

(72) Inventors: Roberto VALDUCCI, Savignano Sul Rubicone (IT); Serozh AVANESSIAN, Villa Verucchio (IT)

(21) Appl. No.: 14/908,747

(22) PCT Filed: Aug. 1, 2014
(86) PCT No.: PCT/EP2014/066599

§ 371 Date: Jan. 29, 2016

(30) Foreign Application Priority Data
Aug. 1, 2013 (IT) FI2013A000184

(51) Int. Cl. A61K 31/403; A61K 47/02; A61K 9/20; A61K 47/38

(52) U.S. Cl. CPC A61K 31/403; A61K 47/38; A61K 47/02; A61K 9/2095; A61K 9/2054; A61K 9/2009

(57) ABSTRACT

Sustained-release Gliclazide tablets devoid of calcium phosphate dibasic, and containing only soluble excipients. The mixing of two high viscosity HPMC with a low viscosity one allows to obtain releases similar to those of the reference products on the market, and it shows also a certain pH dependence.

### FIELD OF THE INVENTION

The present invention relates to a pharmaceutical composition comprising Gliclazide, in particular sustained-release tablets containing Gliclazide.

### STATE OF THE ART

Gliclazide is a second generation hypoglycemic sulfonylurea used in the treatment of diabetes. It is usually administered by oral route in controlled-release formulations capable of making the active ingredient available for absorption.

In WO 00/18373 A1 a controlled-release Gliclazide tablet is described, said tablet comprising 12-40% w/w of Gliclazide, 10-40% w/w of a hydroxypropylmethyl cellulose (HPMC) 4000 cps and HPMC 100 cps mixture, 2-20% w/w of maltodextrin and 35-75% w/w of CaHPO₄ as diluent, wherein % w/w referred to the total weight of the tablet.

The patent WO 2006/123213 refers to a formulation obtained by granulation (dry or wet) of Gliclazide with addition of binders and other excipients. In order to modulate the release of Gliclazide, a mixture of two types of HPMC (HPMC K100LV and HPMC K4M CR) in extra-granular phase is added to this granulate.

The patent WO 2006/061697 describes a formulation containing Gliclazide, with particle size lower than 50 microns, a mixture of two HPMC polymers (HPMC K100LV and HPMC K4M CR) to control the release, and a mono- and/or a disaccharide (lactose) added to the formulation by wet granulation.

The patent WO 2008/062470 describes a formulation containing Gliclazide granulated with HPMC K4M, CaHPO₄, and a solution of PVP K30 in iPrOH, and it does not include any saccharide component.

All the previous formulations have in common the use of calcium phosphate dibasic anhydrous or dihydrate as diluent.

All previous formulations show a substantially pH-independent release kinetics at pH between 4 and 8.

### SUMMARY OF THE INVENTION

The object of the present invention is a Gliclazide sustained-release pharmaceutical composition; said composition comprising:

Gliclazide, as active ingredient;

a mixture of cellulose derivatives, said mixture comprising at least two different derivatives having medium or high viscosity, and at least one derivative having low viscosity;

other diluents, all soluble in water;

wherein medium viscosity means a viscosity not lower than 2000 cps, high viscosity means a viscosity higher than 50000 cps, and low viscosity means a viscosity lower than 100 cps.

The composition object of the invention is devoid of CaHPO₄.

The formulation object of the present patent shows a pH-dependent release kinetics, and in particular it shows a release comparable to that of the leading product of reference (described in EP2103302, and commercially available as Diamicron®) at pH 6.8, while at pH 4.5 and in water, the release profile of the formulation deviates significantly from the results obtained with the official method of analysis at pH 6.8. One may therefore state that this formulation has, unlike the formulations known in the art, a pH dependence.

### DETAILED DESCRIPTION OF THE INVENTION

The pharmaceutical composition according to the invention is for Gliclazide oral administration.

Preferably, the cellulose derivatives are selected from the group consisting of hydroxymethyl cellulose, hydroxyethyl cellulose, hydroxypropyl cellulose and hydroxypropylmethyl cellulose (HPMC). The mixture of cellulose derivatives, according to the invention, preferably has a total viscosity between 6000 and 21000 cps.

Preferably, the mixture of cellulose derivatives is consisting of three HPMC of which:

one HPMC having viscosity higher than 50000 cps, one HPMC having viscosity of between 2000 and 50000 cps, and one HPMC having viscosity lower than 100 cps.

More preferably, the mixture of cellulose derivatives is consisting of:

one HPMC having viscosity higher than 70000 cps, one HPMC having viscosity of between 3000 and 30000 cps, and one HPMC having viscosity lower than 50 cps.

More preferably, the three HPMC are selected from HPMC 5 cps, HPMC 4000 cps, HPMC 15000 cps, and HPMC 100000 cps.

The composition has an active ingredient content between 15% and 20% w/w with respect to the total of the composition, wherein the release is controlled by the polymer matrix consisting of the mixture of cellulose derivatives, preferably HPMC, mixed together in order to obtain a porous matrix and fast hydration. As preferred embodiment, this was obtained by mixing two high or medium viscosity HPMC with a low viscosity HPMC in percentages ranging between 20 and 40% w/w with respect to the total weight of the formulation, more preferably 30% w/w of the finished tablet. Preferably, the low viscosity HPMC is present in amounts ranging between 7% w/w and 15% w/w with respect to the total of the formulation.

Surprisingly, it has been found that the presence of low viscosity HPMC associated with high viscosity HPMC leads to a higher initial hydration of the matrix resulting in a slower early hours release kinetics than the combination of only two HPMC with a viscosity higher than 4000 cps.

Surprisingly, the addition of low viscosity HPMC to a mixture of two medium and/or high viscosity HPMC increases the hydration of the system carrying a synergistic action with other soluble excipients present in the formulation. This effect slows down the release in the first 4 h, after which the release is controlled by the presence of the other two more viscous HPMC.

The absence of calcium phosphate dibasic does not affect the release.

The presence of water-soluble diluents, between 40 and 60% w/w with respect to the total weight of the composition (more preferably 50%), leads to a further increase of the canalization and, therefore, the hydration of the matrix. Preferably said water-soluble diluents are selected from the group consisting of polyalcohols and mixtures thereof; in particular mannitol, maltodextrin, sorbitol, isomalt, and mixtures thereof.

Surprisingly, at pH 6.8, a release identical to the one of the leading product has been obtained without any addition of calcium phosphate dibasic to the composition, and using excipients all soluble. In addition to the active ingredient, the polymers and the soluble diluents, in the formulation also a glidant and a lubricant may be included, in order to improve the workability. Said glidant is preferably chosen from the group consisting of magnesium stearate, sodium stearyl fumarate, stearic acid, or other suitable glidants, or mixture thereof. Said lubricant is preferably chosen from the group consisting of Anhydrous Colloidal Silica, or other suitable lubricants, or mixture thereof. If present, glidant and lubricant percentages are not exceeding 1% w/w with respect to the total of the composition.

In a preferred manner, the composition object of the present invention has the following % w/w composition with respect to the total of the composition:

| | |
|---|---|
| Gliclazide | 15-20%; |
| mixture of cellulose derivatives | 20-40%; |
| water-soluble diluents | 40-60%; |
| glidant | 0-1%; |
| lubricant | 0-1%. |

In a particularly preferred manner, the composition object of the present invention has the following % w/w composition with respect to the total of the composition:

| | |
|---|---|
| Gliclazide | 15-20%; |
| HPMC 5 cps | 7-15%; |
| HPMC 100000 cps | 6-12%; |
| HPMC 4000 cps | 4-8%; |
| Mannitol | 20-30%; |
| Maltodextrin | 20-30%; |
| Magnesium Stearate | 0.30-0.60%; |
| Anhydrous Colloidal Silica | 0.50-1.00%. |

The composition subject-matter of the present invention is preferably in the form of tablets obtained by direct compression or by induction granulation. In a particularly preferred embodiment, the invention relates to 60 mg Gliclazide tablets, said tablets being divisible into two or more dosing fractions. It has been observed that tablets divided into two half-doses show, in turn, a different release profile to the whole tablet but, surprisingly, comparable to the kinetics of other 30 mg Gliclazide formulations known in the art (see EP2103302).

Preferably, the composition object of the present invention may be prepared by mixing Gliclazide to the mixture of cellulose derivatives in order to obtain a mixture to which the water-soluble diluents and finally the glidant and the lubricant are added. The tablets production method may be a direct compression of the mixture of all components described above. In particular, in a preferred embodiment of the invention, the tablets are oblong in shape and equipped with a fracture line to facilitate the divisibility in half.

### BRIEF DESCRIPTION OF THE FIGURES

FIG. 1 shows the dissolution profile of the tablets according to the invention: full dose (60 mg) and half dose (30 mg).

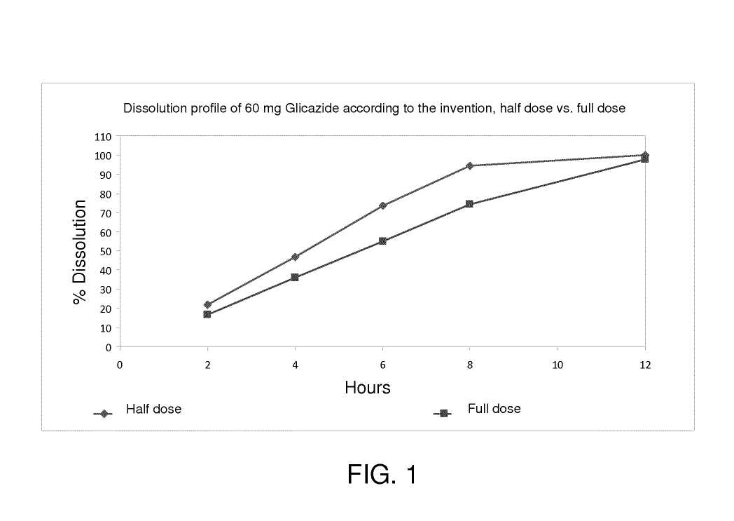

FIG. 2 shows the dissolution profile of the tablets according to the invention in comparison to a reference tablets known in the art.

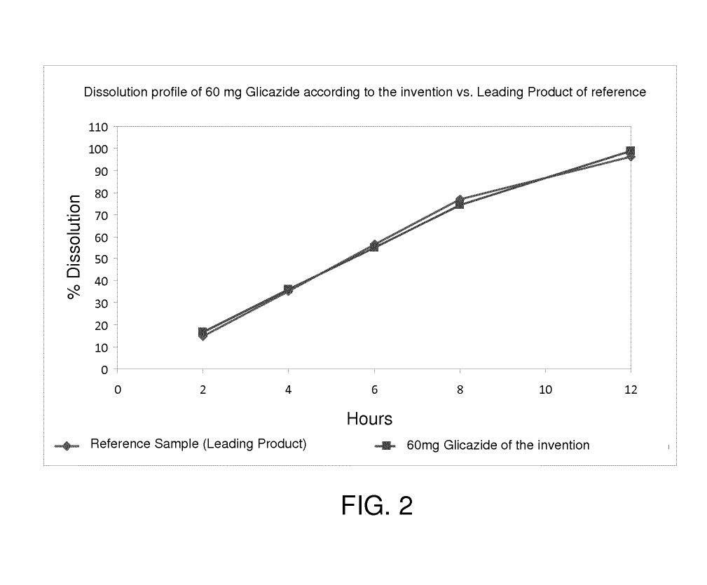

### EXAMPLE 1

### Manufacturing of 60 mg/CPR Gliclazide Divisible Tablets

The percentages of the components used in the formulation are shown in the following table:

| Component | % |
|---|---|
| Gliclazide | 18.7 |
| HPMC 5 cps | 13.5 |
| HPMC 100000 cps | 10.5 |
| HPMC 4000 cps | 6 |
| Mannitol | 25 |
| Maltodextrin | 25 |
| Magnesium Stearate | 0.45 |
| Anhydrous Colloidal Silica | 0.85 |

Gliclazide is added into a high speed granulator together with the three HPMC in order to incorporate in an optimal manner the active ingredient into the polymers that will form the matrix. To this mixture, always into the granulator, Mannitol and Maltodextrin, which will act as diluents and channeling agents, are added. Finally, sieved Anhydrous Colloidal Silica and Magnesium Stearate are added into the bin. The mixture obtained is then compressed by means of a rotary tablet press fitted with oblong punches with a fracture line.

The releases of the tablets obtained with this method are given below.

Comparison of dissolution profiles of half dose of 60 mg Gliclazide according to the invention vs. full dose of 60 mg Gliclazide according to the invention:

Dissolution medium: phosphate buffer pH 6.8, 1000 ml

Apparatus: stationary basket, 50 rpm

Reference sample: half dose of 60 mg Gliclazide tablet

| Hours | R1 | R2 | R3 | R4 | R5 | R6 | R average | DSR |
|---|---|---|---|---|---|---|---|---|
| 2 | 21.5 | 20.9 | 21.0 | 22.4 | 22.3 | 23.9 | 22.0 | 5.1 |
| 4 | 45.9 | 45.9 | 45.4 | 46.0 | 47.1 | 49.9 | 46.7 | 3.6 |
| 6 | 72.5 | 71.8 | 70.4 | 72.6 | 73.5 | 80.4 | 73.5 | 4.8 |
| 8 | 93.8 | 94.4 | 92.0 | 92.9 | 96.4 | 96.5 | 94.3 | 2.0 |
| 12 | 100.1 | 97.6 | 102.2 | 98.1 | 102.9 | 98.4 | 99.9 | 2.2 |

Sample under test: full dose of 60 mg Gliclazide tablet

| Hours | T1 | T2 | T3 | T4 | T5 | T6 | T average | DSR |
|---|---|---|---|---|---|---|---|---|
| 2 | 15.4 | 14.7 | 16.5 | 19.1 | 17.7 | 17.6 | 16.8 | 9.6 |
| 4 | 33.1 | 32.2 | 36.1 | 39.1 | 37.5 | 37.5 | 35.9 | 7.6 |
| 6 | 51.6 | 50.4 | 56.3 | 59.7 | 55.9 | 56.0 | 55.0 | 6.2 |
| 8 | 70.6 | 70.3 | 75.7 | 78.1 | 75.3 | 75.3 | 74.2 | 4.2 |
| 12 | 99.8 | 97.9 | 94.4 | 97.0 | 98.2 | 98.5 | 97.6 | 1.9 |

f₂=43,6

The Similarity Factor or f2 is calculated using the following formula:

f₂=50·log {[1+(1/n·Σ(Rₜ−Tₜ)²]^{-0.5}·100}

wherein:
n=Number of sampling times
Rₜ=Dissolution value of the reference sample at time t
Tₜ=Dissolution value of the sample under test at time t

As it can be seen from the tables, the releases of the full dose and of the half dose are not comparable to each other since the Similarity Factor (f2) is lower than 50.

However, the half dose and full dose taken individually show releases that are comparable to those of the reference products such as Diamicron®, 30 mg and 60 mg respectively.

Comparison of dissolution profiles of 60 mg Gliclazide leading product of reference (Diamicron®) vs. 60 mg Gliclazide Valpharma per tablet:

Dissolution medium: phosphate buffer pH 6.8, 1000 ml

Apparatus: stationary basket, 50 rpm

Reference sample: 60 mg Gliclazide per tablet leading product of reference Diamicron® manufactured by Laboratoires Servier, Batch No.: 889195

| Hours | R1 | R2 | R3 | R4 | R5 | R6 | R7 | R8 | R9 | R10 | R11 | R12 | R average | DSR |
|---|---|---|---|---|---|---|---|---|---|---|---|---|---|---|
| 2 | 13.2 | 13.9 | 14.1 | 13.7 | 14.5 | 14.9 | 18.8 | 12.2 | 16.9 | 15.0 | 16.1 | 14.1 | 14.8 | 12.0 |
| 4 | 32.4 | 34.0 | 34.1 | 34.1 | 33.6 | 33.8 | 41.5 | 30.9 | 41.3 | 36.1 | 38.1 | 33.1 | 35.3 | 9.6 |
| 6 | 53.1 | 56.1 | 55.6 | 55.2 | 54.1 | 54.4 | 63.4 | 50.6 | 64.0 | 58.8 | 61.0 | 52.5 | 56.6 | 7.6 |
| 8 | 74.3 | 77.3 | 75.7 | 75.3 | 72.8 | 72.6 | 83.7 | 72.5 | 85.1 | 79.9 | 80.5 | 72.2 | 76.8 | 5.9 |
| 12 | 96.3 | 95.1 | 97.6 | 95.9 | 93.2 | 90.4 | 99.3 | 95.7 | 99.5 | 97.9 | 96.8 | 96.5 | 96.2 | 2.6 |

Sample according to the invention: Gliclazide 60 mg per tablet, Batch No.: 0000040851

| Hours | T1 | T2 | T3 | T4 | T5 | T6 | T7 | T8 | T9 | T10 | T11 | T12 | T average | DSR |
|---|---|---|---|---|---|---|---|---|---|---|---|---|---|---|
| 2 | 15.4 | 14.7 | 16.5 | 19.1 | 17.7 | 17.6 | 18.5 | 14.6 | 17.5 | 15.3 | 17.9 | 16.9 | 16.8 | 8.9 |
| 4 | 33.1 | 32.2 | 36.1 | 39.1 | 37.5 | 37.5 | 39.3 | 33.0 | 38.2 | 32.8 | 37.4 | 35.5 | 36.0 | 7.2 |
| 6 | 51.6 | 50.4 | 56.3 | 59.7 | 55.9 | 56.0 | 58.8 | 52.7 | 59.1 | 49.5 | 55.9 | 54.0 | 55.0 | 6.2 |
| 8 | 70.6 | 70.3 | 75.7 | 78.1 | 75.3 | 75.3 | 79.5 | 74.5 | 78.1 | 65.3 | 74.5 | 75.1 | 74.4 | 5.3 |
| 12 | 99.8 | 97.9 | 94.4 | 97.0 | 98.2 | 98.5 | 100.4 | 104.5 | 97.0 | 98.2 | 100.0 | 98.3 | 98.7 | 2.5 |

f₂=82,8

The analysis performed at different pHs show that the releases of this formulation are pH-dependent, in fact, at pH 4.5 and in water, releases that are very different from the ones at pH 6.8 are obtained. (see table below)

MEDIUM

| | |
|---|---|
| pH 4.5/1000 ml | Water/1000 ml |

APPARATUS

| | Paddle 50 rpm + | Paddle 50 rpm + |
|---|---|---|
| | Stationary Basket | Stationary Basket |
| TIME | average | average |
| 2 h | 12.9 | 15.6 |
| 4 h | 29.9 | 36.2 |
| 6 h | 45.3 | 51.6 |
| 8 h | 55.3 | 60.8 |
| 10 h | 57.6 | 65.3 |
| 12 h | 59.8 | 66.6 |
| 16 h | 60.4 | 68.0 |
| 20 h | 60.4 | 68.8 |
| 24 h | 60.9 | 68.8 |

1-13. (canceled)

14. A modified-release pharmaceutical composition comprising;
Gliclazide, in admixture with a mixture of at least three cellulose derivatives, at least two of which are different derivatives have medium or high viscosity and at least one of which has low viscosity; and
the rest of the composition being at least one additional diluent, wherein said additional diluents are all soluble in water;
said composition being devoid of CaHFO₄.

15. The composition according to claim 14, wherein said cellulose derivatives are selected from the group consisting of hydroxymethyl cellulose, hydroxyethyl cellulose, hydroxypropyl cellulose and hydroxypropylmethyl cellulose (HPMC).

16. The composition according to claim 15, wherein the mixture of cellulose derivatives consists of three HPMCs, wherein one HPMC has a viscosity higher than 50000 cps, one HPMC has a viscosity from 2000 to 50000 cps, and one HPMC has a viscosity lower than 100 cps.

17. The composition according to claim 16, wherein the HPMCs are chosen from the group consisting of HPMC Sops, HPMC 4000 cps, HPMS 15000 cps and HPMC 100000 cps.

18. The composition according to claim 14, wherein said mixture of cellulose derivatives is present at a percentages of from 20% to 40% why with respect to the total weight of the composition.

19. The composition according to claim 14, wherein said water-soluble diluents are selected from the group consisting of polyalcohols and mixtures thereof.

20. The composition of claim 19, wherein said polyalcohol is selected in the group consisting of mannitol, maltodextrin, sorbitol, isomalt and mixtures thereof.

21. The composition according to claim 14, comprising a glidant and/or a lubricant.

22. The composition according to claim 21, wherein the glidant is magnesium stearate, sodium stearyl fumarate, stearic acid or mixtures thereof, and the lubricant is anhydrous colloidal silica.

23. The composition according to claim 14, consisting of the following % w/w composition with respect to the total composition:

| | |
|---|---|
| Gliclazide | 15-20%; |
| mixture of cellulose derivatives | 20-40%; |
| water-soluble diluents | 40-60%; |
| glidant | 0-1%; |
| lubricant | 0-1%. |

24. The composition according to claim 14 in tablet form.

25. The composition according to claim 14, wherein said tablet contains 60 mg Gliclazide, said tablet being divisible into two or more dosing fractions.

26. A method of producing a composition according to claim 14, comprising adding Gliclazide to a mixture of cellulose derivates in order to obtain a first mixture and adding a water-soluble diluent to said first mixture to obtain a second mixture.

27. The method of claim 26, further comprising adding a glidant and/or a lubricant to said second mixture.

28. The method according to claim 26, wherein the mixture of all the components is subjected to direct compression or to induction granulation.
